(19) United States
(12) Patent Application Publication
(10) Pub. No.: US 2005/0113270 A1
(43) Pub. Date: May 26, 2005

(54) Soap apparatus with embedded scrubbing element

(76) Inventors: Neil G. Stockman, City of Des Moines, WA (US); Skye Wollenberg, City of Seattle, WA (US)

Correspondence Address: DORSEY & WHITNEY LLP;INTELLECTUAL PROPERTY DEPARTMENT, SUITE 3400, 1420 FIFTH AVENUE, SEATTLE, WA, 98101, US

(21) Appl. No.: 10/718,991

(22) Filed: Nov. 21, 2003

(51) Int. Cl. A61K 7/50

(52) U.S. Cl. 510/141

(57) ABSTRACT

The present invention relates to an apparatus and method to form a soap apparatus having an embedded scrubbing element. In one embodiment, a scrubbing soap bar includes a scrubbing element having a filamentous network with internal void regions and a soap material that substantially surrounds the scrubbing element and at least partially fills the void regions within the scrubbing element. In another embodiment, a method includes forming a scrubbing element from a non-woven and porous material, and infiltrating the non-woven and porous material with a soap material to form the soap bar. In still another embodiment, a method includes positioning a scrubbing element in a mold configured to receive the scrubbing element, adding a soap material to the mold to form a solid bar that encapsulates the scrubbing element, and removing the solid bar from the mold.

### TECHNICAL FIELD

The present invention relates generally to implements used to clean undesired foreign matter from a variety of surfaces, and in particular to a soap apparatus having an embedded scrubbing element.

### BACKGROUND OF THE INVENTION

Soap products in the form of a bar have long been in use for a variety of cleansing purposes. The soap product may be comprised of a variety of specific compositions, but is usually formed by saponifying a fatty acid with a base, which generally includes a metal hydroxide or a carbonate, to yield a surface-active material that is basic. When the soap product is used with water in the conventional manner, the resulting mixture is particularly effective in dissolving oils and greases. The resulting mixture is generally slippery, however, which inhibits the ability of the user to transfer the soap to the surface to be cleaned, and may also inhibit the ability of the user to dislodge foreign material from the object to be cleansed.

Accordingly, a wide variety of implements are often used in conjunction with the soap product in order to enhance the cleansing effect of the soap product. For example, a cloth, a sponge or even a brush are commonly used to transfer the soap solution to a surface to be cleansed. The foregoing implements also mechanically dislodge foreign matter from the surface while the surfactant properties of the soap. As a result, effective cleansing in many circumstances is obtained only when the foregoing implements are available.

In order to provide a soap product that combines the surfactant capabilities of a soap with a mechanical abrasive capability, various known products also include abrasive particles such as pumice, or other similar abrasive materials in order to enhance the ability of the soap product to dislodge foreign matter. Although these products possess an enhanced ability to remove foreign matter, they do not have the ability to remove foreign matter in small recesses, such as crevices. For example, when soap products having an abrasive component are used for washing hands, such products are particularly ineffective in removing foreign matter from beneath fingernails.

Other prior art products employ an embedded structure in a soap product. For example, U.S. Patent No. to Ruff discloses a scrubbing soap bar having a length of a thin, fine mesh netting embedded in the soap bar. Since the mesh netting is “wadded” when the bar is formed, the netting may assume a number of various shapes and configurations within the bar. As a consequence, a product having uniformly consistent cleansing properties is generally not obtainable. U.S. Pat. No. 6,171,007 B1 to Hsu discloses a net embedded in a cake of a soap material. The disclosed net is comprised of a perforated sheet of material that is formed into an envelope, which is then cast into a soap bar. Although the disclosed net avoids the particular shortcoming present in the foregoing device, the: ability of a perforated sheet to remove foreign matter by mechanical action is limited. U.S. Pat. No. 4,050,825 to Stein discloses a soap bar having a brush element integrally formed in the bar. Although the brush is capable of cleansing a surface with greater effectiveness, the integral brush contributes significantly to the overall cost of the soap bar.

Accordingly, there is a need in the art for a soap product having an embedded scrubbing device that offers enhanced cleansing capabilities that is relatively inexpensive to manufacture.

### SUMMARY OF THE INVENTION

The present invention relates generally to an apparatus used to clean undesired foreign matter from a variety of surfaces, and in particular to a soap apparatus having an embedded scrubbing element. In one aspect of the invention, a scrubbing soap bar includes a scrubbing element having a filamentous network with internal void regions and a soap material that substantially surrounds the scrubbing element and at least partially fills the void regions within the scrubbing element. In another aspect of the invention, a method of manufacturing a soap bar having a scrubbing element includes forming a scrubbing element from a non-woven and porous material, and infiltrating the non-woven and porous material with a soap material to form the soap bar. In still another aspect, a method of manufacturing a soap bar having a scrubbing element includes positioning a scrubbing element in a mold configured to receive the scrubbing element, adding a soap material to the mold to form a solid bar that encapsulates the scrubbing element, and removing the solid bar from the mold.

### BRIEF DESCRIPTION OF THE DRAWINGS

FIG. 1 is an isometric view of a soap bar having an embedded scrubbing element according to an embodiment of the invention.

FIG. 2 is an isometric view of the embedded element for the embodiment shown in FIG. 1.

FIG. 3 is a cross sectional view of a step in a method for manufacturing a soap bar according to another embodiment of the invention.

FIG. 4 is a cross sectional view of another step in a method for manufacturing a soap bar according to another embodiment of the invention.

FIG. 5 is a cross sectional view of a still another step in a method for manufacturing a soap bar according to another embodiment of the invention.

### DETAILED DESCRIPTION OF THE INVENTION

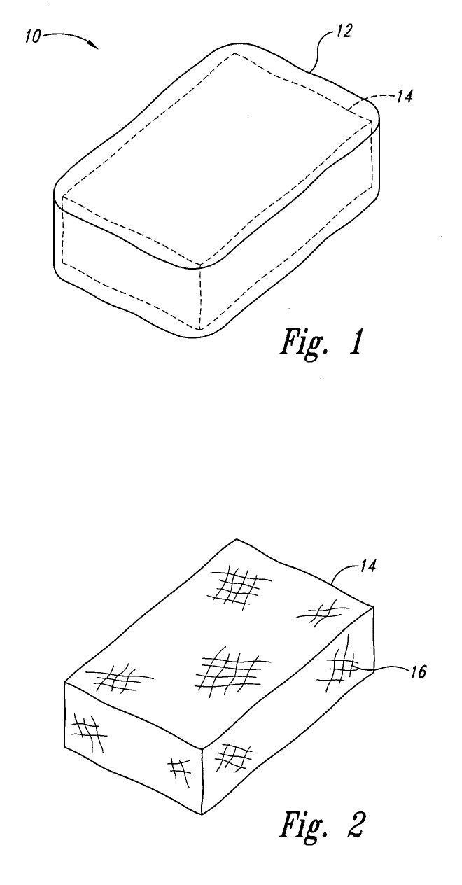

The present invention is generally directed to implements used to clean undesired foreign matter from a variety of surfaces, and in particular to a soap bar having an embedded scrubbing element. Many of the specific details of certain embodiments of the invention are set forth in the following description and in FIGS. 1 to 4 to provide a thorough understanding of such embodiments. One skilled in the art will understand, however, that the present invention may be practiced without several of the details described in the following description. Moreover, in the description that follows, it is understood that the figures related to the various embodiments are not to be interpreted as conveying any specific or relative physical dimension. Instead, it is understood that specific or relative dimensions related to the embodiments, if stated, are not to be considered limiting unless the claims expressly state otherwise.

FIG. 1 is an isometric view of a soap bar 10 according to an embodiment of the invention. The soap bar 10 includes a solid soap material 12 that encases a scrubbing element 14. The soap material 12 may include well-known materials formed by the saponification of organic fat stocks with an alkali, and may include other synthetic surfactants, such as fatty alcohol ether sulfates or fatty acid isethionates. Alternately, the soap material 12 may be comprised of various known synthetic detergent materials. In either case, the soap material 12 may also include additional additives, such as lanolin, lecithin, various vegetable oils, vitamins including vitamin E, partial glycerides and other fat-like substances that are effective in moisturizing cleansed skin, various antioxidants such as ascorbyl palitate or tocopherol for preventing rancidity in the soap material 12, as well as various dyes and perfumes to impart a desired color and scent.

Referring still to FIG. 1 and also now to FIG. 2, the scrubbing element 14 is a generally filamentous mass of synthetic or organic fibers 16 that is formed in a cubical or rectangular cross-sectional shape to generally conform to the conventional shape of a soap bar, although other shapes, including ellipsoids, oblate ellipsoids and even spherical shapes may be used. In a particular embodiment, the scrubbing element 14 is comprised of a durable, three-dimensional, non-woven nylon filament network generally known as SCOTCH BRITE, available from the Minnesota Mining and Manufacturing Co. (3M) of St. Paul, Minn., although other similar materials may also be used. In other aspects, the scrubbing element 14 may be comprised of various natural filamentous materials such as wool, cotton or other similar fine, fibrous substances, or even the fibrous portion of various vascular plants, such as the well-known tropical dishcloth gourd of the Curcurbitaceae family. The scrubbing element 14 may also be comprised of still other materials. For example, natural or synthetic sponges may be used. Additionally, fibrous metallic materials, known generally as “steel wool”, and other similar materials may also be used.

Still referring to FIGS. 1 and 2, the scrubbing element 14 is positioned within the soap material 12 so that the scrubbing element 14 substantially extends throughout a volume of the soap bar 10. Further, the scrubbing element 14 is encased within the soap material 12 so that the soap material 12 also substantially occupies and fills void portions present in the scrubbing element 14.

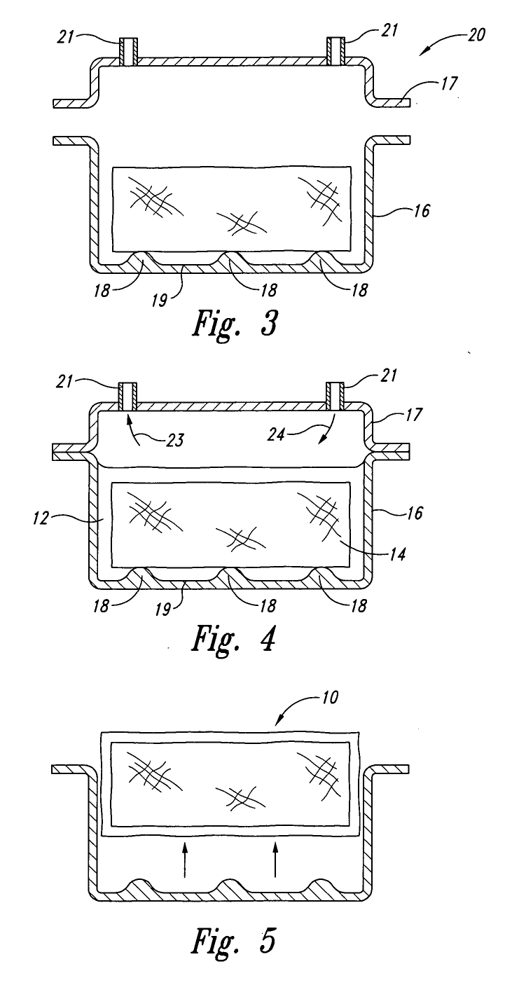

FIG. 3 is a cross-sectional view illustrating a step in a method 20 of manufacturing the soap bar 10 of FIG. 1. The scrubbing element 14 is positioned in a mold 16 that is suitably sized to receive the scrubbing element 14. The mold 16 may additionally include one or more ribs 18 integrally formed on a lower surface 17 of the mold 16 to position the scrubbing element 14 away from the lower surface 17 so that the soap material 12 may substantially surround the soap bar 10 during the formation process, as will be described in further detail below. The mold 16 may also include a removable cover 17 that permits the scrubbing element 14 to be enclosed in a hermetically sealed enclosure when the cover 17 is fixedly attached. The cover 17 also includes one or more ports 21 that extend through the cover 17 to permit air to be withdrawn from the enclosure, and further to permit the soap material 12 to be supplied to the mold 16.

FIG. 4 is a cross-sectional view illustrating another step in a method 20 of manufacturing the soap bar 10 of FIG. 1. With the cover 17 of the mold 16 attached, air 23 may be evacuated from the mold 16 through the port 21 prior to supplying the soap material 12 to the mold. The air 23 may be removed from the mold 16 by a vacuum pump or by other similar means so that the air 23 is at least partially removed from the void portions in the scrubbing element 14. Following the removal of the air 23 from the mold 16, the soap material 12 may be supplied to the mold 16 as a semi-liquid stream 24. Since the air 23 has at least partially been removed from the scrubbing element 14, the soap material 12 fully permeates the void portions in the scrubbing element 14 during the forming process. After the semi-liquid material 24 sets in the mold the soap bar 10 is formed.

FIG. 5 is a cross-sectional view illustrating a final step in a method 20 of manufacturing the soap bar 10 of FIG. 1. After the soap bar 10 is formed in the mold 16 as previously described, the cover 17 (as shown in FIGS. 3 and 4) is removed, and the soap bar 10 is removed from the mold 16. Although the foregoing method describes forming the soap bar 10 by at least partially evacuating the mold 16 so that air is removed from the scrubbing element 14, it is understood that the soap bar 10 may also be formed by allowing the soap material 12 to gradually permeate into the scrubbing element 14 without the assistance of a partial vacuum within the mold 16.

The foregoing embodiments of the invention offer significant advantages over the prior art. For example, since the scrubbing element 14 extends throughout the soap bar 10, the scrubbing element 14 it is not detachable from the soap bar 10, particularly as the soap material 12 gradually dissolves as the soap bar 10 is used. The filamentous structure of the scrubbing element 14 advantageously permits the soap bar 10 to cleanse objects having recesses and crevices with greater effectiveness than other prior art soap bars since the filaments possess the ability to reach into recesses and crevices.

From the foregoing it will be appreciated that, although specific embodiments of the invention have been described herein for purposes of illustration, various modifications may be made without deviating from the spirit and scope of the invention. For example, certain features shown in the context of one embodiment of the invention may be incorporated into other embodiments as well. Accordingly, the invention is not limited by the foregoing description of embodiments except as by the following claims.

1. A scrubbing soap bar, comprising:
a scrubbing element having a filamentous network with internal void regions; and
a soap material that substantially surrounds the scrubbing element and at least partially fills the void regions.

2. The scrubbing soap bar of claim 1, wherein the scrubbing element is further comprised of a non-woven network of polymer fibers.

3. The scrubbing soap bar of claim 1, wherein the scrubbing element is further comprised of a non-woven network of organic fibers.

4. The scrubbing soap bar of claim 1, wherein the scrubbing element is further comprised of a non-woven network of metallic fibers.

5. The scrubbing soap bar of claim 2, wherein the non-woven network of polymer fibers includes a non-woven network of fibers comprised of nylon.

6. The scrubbing soap bar of claim 4, wherein the non-woven network of metallic fibers includes a non-woven network of fibers comprised of a ferrous material.

7. The scrubbing soap bar of claim 4, wherein the non-woven network of metallic fibers includes a non-woven network of fibers comprised of a non-ferrous material.

8. The scrubbing soap bar of claim 1, wherein the scrubbing element further comprises a natural or a synthetic sponge material.

9. The scrubbing soap bar of claim 1, wherein the soap bar is further comprised of a material formed by the saponification of an organic fat with an alkali.

10. The scrubbing soap bar of claim 1, wherein the soap material is further comprised of a synthetic detergent material.

11. A method of manufacturing a soap bar having a scrubbing element, comprising:
forming a scrubbing element from a non-woven and porous material; and
infiltrating the non-woven and porous material with a soap material to form the soap bar.

12. The method of claim 11, wherein forming a scrubbing element further comprises forming the scrubbing element from a non-woven network of polymer fibers.

13. The method of claim 11, wherein forming a scrubbing element further comprises forming the scrubbing element from a non-woven network of organic fibers.

14. The method of claim 11, wherein forming a scrubbing element further comprises forming the scrubbing element from a non-woven network of metallic fibers.

15. The method of claim 11, further comprising positioning the scrubbing element in a mold.

16. The method of claim 11, wherein infiltrating the non-woven and porous material scrubbing element further comprises enclosing the scrubbing element in a hermetically sealed mold and at least partially evacuating the mold before infiltrating the soap material into the mold.

17. The method of claim 11, wherein infiltrating the non-woven and porous material with a soap material further comprises infiltrating a material formed by the saponification of an organic fat with an alkali.

18. The method of claim 11, wherein infiltrating the non-woven and porous material with a soap material further comprises infiltrating a synthetic detergent material.

19. A method of manufacturing a soap bar having a scrubbing element, comprising:
positioning a scrubbing element in a mold configured to receive the scrubbing element;
adding a soap material to the mold to form a solid bar that encapsulates the scrubbing element; and
removing the solid bar from the mold.

20. The method of claim 19, wherein positioning a scrubbing element in a mold further comprises positioning a non-woven network of polymer fibers into the mold.

21. The method of claim 19, wherein positioning a scrubbing element in a mold further comprises positioning a non-woven network of organic fibers into the mold.

22. The method of claim 19, wherein positioning a scrubbing element in a mold further comprises positioning a non-woven network of metallic fibers into the mold.

23. The method of claim 19, further comprising permeating the scrubbing element with the soap material.

24. The method of claim 23, wherein permeating the scrubbing element with the soap material further comprises enclosing the scrubbing element in a hermetically sealed mold and at least partially evacuating the mold before adding the soap material to the mold.

25. The method of claim 19, wherein adding a soap material to the mold further comprises adding a soap material to the mold in a semi-liquid state; and curing the semi-liquid material to form the solid bar.

26. The method of claim 19, wherein adding a soap material to the mold further comprises adding a material formed by the saponification of an organic fat with an alkali.

27. The method of claim 19, wherein adding a soap material to the mold further comprises adding a synthetic detergent material to the mold.
